(19) United States
(12) Patent Application Publication
(10) Pub. No.: US 2011/0059124 A1
(43) Pub. Date: Mar. 10, 2011

(54) THE QUALITY CONTROL METHOD AND APPLICATION OF A KIND OF GANODERMA LUCIDUM SPORE OIL FAT EMULSION

(75) Inventors: Lulin Chen, Guangzhou city (CN); Fahuan Ge, Guangzhou city (CN); Wenjun Chen, Guangzhou city (CN); Ronghua Zeng, Guangzhou city (CN); Genghai Meng, Guangzhou city (CN); Aishao Zhang, Guangzhou city (CN); Qingshui Lai, Guangzhou city (CN); Zhaojian Jiang, Guangzhou city (CN)

(73) Assignee: Guangzhou Hanfang Natural Medicine Research & Development Co.Ltd, Guangzhou city Guangdong (CN)

(21) Appl. No.: 12/918,949

(22) PCT Filed: Feb. 26, 2008
(86) PCT No.: PCT/CN2008/000396

§ 371 Date: Nov. 23, 2010

(51) Int. Cl. A61K 36/074; A61P 35/00; G01N 30/02

(52) U.S. Cl. 424/195.15; 73/61.58

(57) ABSTRACT

This invention discloses a kind of quality control method and application of Ganoderma lucidium spore oil fat emulsion. Which includes Ganoderma lucidium spore oil 2˜25%, emulsifier 0.5˜10%, isosmotic agent 0.2˜5%, the remaining content is water and the final pH of Fat emulsion is adjusted to 6˜9. The quality control method of this invention can accurately determine the content of 1,2-oleic-3-palmitic triglyceride and glycerol trioleate in the preparation, and accurately determine the ergosterol content, which serves as the method and basis for quality control of this product. Fingerprints are utilized to grasp the product quality from the overall characteristics of Chromatogram. This invention has clearly defined active ingredients, with excellent bioactivity, and is capable of tumor treatment, while improving the organism's immunity, as well as the life quality of tumor patients undergoing radiotherapy and chemotherapy, and relieving the drug toxicity after treatment. This invention is particularly suitable for both intra-arterial injection and intravenous injection, enabling the Ganoderma lucidium spore oil to directly gain access to the human blood circulation, with short time to onset and complete absorption as well as high security, reliable quality, and low drug toxicity.

### BACKGROUND OF THE INVENTION

1. Technical Field

This invention involves the field of Ganoderma Lucidum Spore Oil Technology, specifically involving the Fat emulsion of Ganoderma Lucidum Spore Oil and its quality control technology and medical application.

2. Description of Related Art

Since the 1980s, Japan has initiated the foundation research in the anti-tumor aspects of Ganoderma; by the 1990s, US, Japan and other countries have applied Ganoderma to clinical practices, particularly in the treatment of cancer and AIDS, attracting wide attention from International researchers. Ganoderma is the common name for fungus G.lucidum.karst and G.japonicumLloyd in Ganoderma Genus of the Polyporaceae Family, with the efficacy of supporting healthy energy, etc, referred to as the top grade in the Herbal Classics. Ganoderma lucidium spore is the ultrafine spores projected from the pileus of Ganoderma lucidium during the mature stage, as the germ cells of Ganoderma lucidium, containing all the genetically active materials of Ganoderma lucidium, whose medical value is attracting more and more attention. The Chemical Ingredients of Ganoderma lucidium spore are very complicated, with the following categories: Fatty Acids, Steroids, Triterpenoids, alkaloids, lactone, proteins and amino-acids, Glycopeptides, Vitamins, Carotene, and inorganic ions, etc. Modern Pharmacological Research indicated that Ganoderma lucidum spore oil is the major ingredient in Ganoderma lucidium spore for combating tumor cells. This research demonstrated that the main ingredients of Ganoderma lucidium spore oil were Oil esters, fatty acids, Ganoderma lucidium acids and ergosterol, as well as many other ingredients, through research and testing analysis for Ganoderma lucidium spore oil.

At present, there are Glossy Ganoderma Spore Oil Soft Capsule with Ganoderma lucidium oil as the main ingredient in the market, applicable for the inhibition and prevention of tumor, as well as to strengthen the immunity for recovery after tumor surgery, etc. However it is in oral dosage form, which is absorbed by the gastrointestinal tract after administration, incapable for direct access to human blood circulation. Ganoderma lucidium spore oil is non-water soluble, causing considerable difficulty for the preparation of common injection. However this problem can be solved through the preparation of Fat emulsion of Ganoderma lucidium spore oil, and the title of the invention for application as

Chinese Patent before was “Fat emulsion of Ganoderma lucidium spore oil”, with the patent number of ZL200410051661.3, which precisely solved this problem. The Chinese Invention Patent with Patent Application No. 200510068335.8 entitled “Intravenous Emulsion Injection of Ganoderma lucidium spore oil and its preparation method” has also disclosed a kind of Intravenous Emulsion Injection of Ganoderma lucidium spore oil. However, Fat emulsion of Ganoderma lucidium spore oil disclosed in these two patents including Fatty oil or Injection Oil, suffered from the deficiency of complicated technique, without clearly defined active ingredients, as well as without further presentation of refining technique for Ganoderma lucidium spore oil Injection as well as chemical ingredients of Fat emulsion and the Quality Control Methods, etc.

There are more and more products related to Ganoderma lucidium spore in the market, such as Ganoderma lucidium spore oil, and Ganoderma lucidium spore and their preparations including Glossy Ganoderma Spore Oil Soft Capsule, Fat emulsion Injection of Ganoderma lucidium spore oil, Fat emulsion Oral Dose of Ganoderma lucidium spore, Ganoderma lucidium spore Capsule, Ganoderma lucidium spore tablets, Ganoderma lucidium spore powder, etc, however it is difficult to tell the fake from the real, and hard to evaluate for quality. There have been quite a few literature reports concerning the quality control method for Ganoderma lucidium spore oil, and the applicant himself has also applied for several invention patents concerning quality control method for Ganoderma lucidium spore oil; however there have been no relevant reports concerning the quality control method for Fat emulsion of Ganoderma lucidium spore oil.

### BRIEF SUMMARY OF THE INVENTION

The purpose of the invention is to overcome the deficiency of Fat emulsion of Ganoderma lucidium spore oil, to provide a kind of Fat emulsion of Ganoderma lucidium oil with clearly defined active ingredients and strong physiological activity.

Another purpose of this invention is to provide a quality control method for the Fat emulsion of Ganoderma lucidium spore oil mentioned above.

The further purpose of the invention is to provide the application of Fat emulsion of Ganoderma lucidium spore oil in the preparation of medicines for the treatment of tumors while improving immunization, and reducing the toxicity of chemotherapy drugs.

In order to realize the goals mentioned above, this invention applies the following technology scheme:

A kind of Fat emulsion of Ganoderma lucidium spore oil, composed of the following ingredients with the following weight percentage:

Ganoderma lucidium spore oil: 2˜25%;

Emulsifier: 0.5˜10%;

Isosmotic agent: 0.2˜5%;

The remaining content is water; and the final pH of Fat emulsion is adjusted to 6˜9.

In the Fat emulsion of Ganoderma lucidium spore oil mentioned above, the aforesaid Ganoderma lucidium spore oil adopts Ganoderma lucidium spore oil after refining, with the refining method as: Centrifuge the Ganoderma lucidium spore oil to eliminate the water content, add into adsorbent of 0.5%˜10% of Ganoderma lucidium spore oil weight, and then agitate till homogeneity, heated to 40˜70° C., keep the temperature constant for 20 to 40 minutes, then centrifuge, after film filtration, refined Ganoderma lucidium spore oil is obtained; the aforesaid adsorbents include one or several of the following: activated carbon, silica gel, Alumina N-neutral, diatomaceous earth, and carclazyte as the mixture.

In the Fat emulsion of Ganoderma lucidium spore oil mentioned above, the aforesaid emulsifiers are optimized to one or several of the following: Soybean Lecithin, Yolk lecithin, pluronic, polyglycerol palmitic diol or alginates as mixture. Among which the best is Yolk lecithin or Soybean Lecithin.

In the Fat emulsion of Ganoderma lucidium spore oil mentioned above, the aforesaid Isosmotic agent is optimized to one or several of the following: glycerin, glucose, Xylitol, maltose or Sorbitol as the mixture, the best of which is glycerin. The function of Isosmotic agent is to make the isosmotic pressure of preparation approaching the physiological isosmotic pressure of the human body.

The first quality control method for Fat emulsion of Ganoderma lucidium spore oil mentioned above adopts HPLC to determine the content of 1,2-oleic-3-palmitic triglyceride and/or Glycerol Trioleate. For every 1 g of Fat emulsion of Ganoderma lucidium spore oil, there is 2 mg to 62.5 mg of 1,2-oleic-3-palmitic triglyceride and/or 1.6 mg to 50.0 mg Glycerol Trioleate.

HPLC mentioned above is carried out pursuant to the following Chromatographic conditions: With octadecyl silane as the bulking agent, with the binary or ternary mixture of the three solvents acetonitrile, isopropanol, dichloromethane at any ratio as the mobile phase, the flow rate of mobile phase is 0.5˜2.0 ml/min; and then detected with evaporative light-scattering detector or differential refractive index detector; the number of theoretical plates shall be calculated pursuant to the peaks of 1,2-oleic-3-palmitic triglyceride or Glycerol Trioleate, both of which shall be no lower than 2000; with the chromatographic column temperature between 10 and 50° C.

The second quality control method for Fat emulsion of Ganoderma lucidium spore oil mentioned above adopts HPLC to determine the content of ergosterol in the product and the content of ergosterol in every 1 g of Fat emulsion of Ganoderma lucidium spore oil is 0.04 mg˜7.5mg. Ergosterols are the characteristic ingredients in fungus plants, since the Ganoderma lucidium spore oil is the only fungus plant oil known at present, therefore, ergosterols could be treated as the characteristic ingredients that distinguish Ganoderma lucidium spore oil from other plant oil.

The chromatographic conditions are: With octadecyl silane as the bulking agent, with methanol, ethanol, acetonitrile, methanol water solution, ethanol water solution or acetonitrile water solution as the mobile phase, or with ternary or quaternary mixture of methanol, ethanol, acetonitrile and water as the mobile phase, or with the mixture of tetrahydrofuran and water as the mobile phase, and the volume ratio between tetrahydrofuran and water is 75:25; with the detecting wavelength of 280±2 nm; the number of theoretical plates shall be calculated pursuant to ergosterol peak, no lower than 2000.

The third kind of quality control method for Fat emulsion of Ganoderma lucidium spore oil adopts HPLC, through comparison between several batches of chromatogram for Fat emulsion of Ganoderma lucidium spore oil, and the standard fingerprints for Fat emulsion of Ganoderma lucidium spore oil, constituted by common characteristic peaks. The chromatographic conditions for the aforesaid HPLC are optimized to: with octadecyl silane as the bulking agent for Chromatographic column; with acetonitrile, isopropanol as the mobile phase, and the volume ratio between them is 53:47; with the evaporative light-scattering detector; control: with Glycerol Trioleate as the control;

In the fingerprint of Fat emulsion of Ganoderma lucidium spore oil, there are 15 common peaks, among which there are 4 fingerprint peaks covering more than 5% of total peak area respectively, with the relative retention time of Glycerol Trioleate chromatogram peak as 1, calculates the relative retention time of other chromatogrm peaks as well as the relative peak area, and the aforesaid four fingerprint peaks are respectively peak 9, with the average retention time RT as 0.778, and the relative peak area is between 9.54 and 15.36%; peak 10, with the average retention time RT as 0.832, and the relative peak area is between 5.76% and 9.43%; peak 11, with the average retention time RT for Glycerol Trioleate as 1.000, and the relative peak area is between 22.29 and 27.80%; peak 12, with the average retention time RT as 1.075, and the relative peak area is between 26.82% and 37.76%.

The pH value of Fat emulsion of Ganoderma lucidium spore oil described in this invention could be adjusted with NaOH or Phosphate Buffer solution during the process of preparation. The final pH value is between 6 and 9.

Other conventional quality indices for Fat emulsion of Ganoderma lucidium spore oil described could be determined with the already known method in this field, among which: the diameters of emulsion particles are evenly distributed between 100 and 500 nm; with less than 1% of emulsion particles with a diameter over 1 mm, and it is forbidden to detect emulsion particles with a diameter over 5 mm.

Peroxide value: lower than 2 mmol/kg.

Bacterial endotoxin: for every 1 ml of Fat emulsion, the endotoxin content shall be lower than 0.5 EU.

The Fat emulsion of Ganoderma lucidium spore oil in this invention has excellent pharmacological activity, enabling the separate or mixed administration with other medicines, to prepare the medications for the treatment of tumors while improving immunization, and reducing the toxicity of chemotherapy drugs. The Fat emulsion of Ganoderma lucidium spore oil is particularly suitable for both intra-arterial injection and intravenous injection, enabling the Ganoderma lucidium spore oil to directly gain access to the human blood circulation. The best dosage form is the Fat emulsion for injection.

Compared with existing technologies, this invention has the following beneficial results:

1. The Ganoderma lucidium spore oil Fat emulsion has clearly defined active ingredients, with excellent bioactivity, and could be administered separately or combined with other anti-tumor medicines for the treatment of tumors and improving immunity of organism, for tumor patients undergoing radiotherapy and chemotherapy, it could improve their Quality of Life and reduce the drug toxicity after treatment.

2. The Fat emulsion of Ganoderma lucidium spore oil is particularly suitable for both intra-arterial injection and intravenous injection, enabling the Ganoderma lucidium spore oil to directly gain access to the human blood circulation, with short time to onset and complete absorption.

3. The Ganoderma lucidium spore oil Fat emulsion in this invention is a product with high security, reliable quality and low toxicity.

4. The raw materials for the preparation of Ganoderma lucidium spore oil Fat emulsion in this invention is easy to obtain, with a simple process, suitable for the preparation with conventional manufacturing equipment and process, and could be put into mass production in large scale in pharmaceutical plants with GMP manufacturing capacity for injections.

5. The quality control method of this invention can accurately determine the content of 1,2-oleic-3-palmitic triglyceride and glycerol trioleate in the preparation, which serves as the method and basis for quality control of this product, with a simple method, and high operability as well as high repeatability.

6. The quality control method of this invention can accurately determine the content of ergosterol in the preparation, which serves as the method and the basis for quality control of this product, with high operability, high repeatability and strong specificity.

7. The quality control method of this invention utilizes fingerprints, with a simple method, high stability and precision, and high repeatability, it is easy to master, and could grasp the type and quality conditions of Ganoderma lucidium spore oil Fat emulsion from the overall characteristics of chromatogram, and provide a brand new method for the quality control and authentication of Ganoderma lucidium spore oil Fat emulsion.

### BRIEF DESCRIPTION OF THE SEVERAL VIEWS OF THE DRAWINGS

FIG. 1. High Performance Liquid Chromatogram at 0 to 60 minutes for triglyceride control;

FIG. 2. Standard fingerprint at 0 to 60 minutes for Ganoderma lucidium spore oil Fat emulsion;

FIG. 3. Fingerprint at 0 to 60 minutes for 10 batches of Ganoderma lucidium spore oil Fat emulsion.

### DETAILED DESCRIPTION OF THE INVENTION

### Embodiments 1˜6

Refining of Ganoderma lucidium spore oil: Centrifuge the Ganoderma lucidium spore oil to eliminate the water content, and add into activated carbon covering 5% of the weight of Ganoderma lucidium spore oil, to agitate till homogeneity, and then heated to 50° C., keeping the temperature constant for 30 minutes, then centrifuge, through precision filtration, the refined Ganoderma lucidium spore oil can be obtained.

Preparation method: Under the condition of nitrogen, add the emulsifier into refined Ganoderma lucidium spore oil, pounding at high speed till the emulsifier dissolves completely; with constant water bath at 50° C. and under the condition of high speed dispersion and emulsifying at 12000 rpm, add the oil phase slowly into the isosmotic water solution, and blend till homogeneity; adjust the pH between 6 and 9 with NaOH; immediately put the prepared initial emulsion into the homogenizer, and adjust the homogenizing pressure to 60 Mpa, and homogenize 8 times, at the temperature of 60° C., after which filtrate with microfiltration membrane, and then fill into an infusion bottle, add nitrogen, and seal with butyl rubber stopper and aluminum cap; after sterilization, the end product is obtained. The product manufactured complies with the provisions of the aforesaid quality control, as well as relevant preparation standard in pharmacopeia.

Consulting the aforesaid preparation method, the formula of Embodiment 1˜6 are indicated in Table 1, and the aforesaid mass percentage in Table 1 applies the weight percentage.

TABLE 1
The Formula of Embodiment 1~6

| | Refined | | | | |
|---|---|---|---|---|---|
| | Ganoderma | | | | |
| | lucidium | | | | |
| | spore oil | Emulsifier | Isosmotic agent | Water | pH |
| Embodiment 1 | 2% | Yolk lecithin 3% | Glycerin 5% | 90% | 6.5 |
| Embodiment 2 | 25% | Pluronic 5% | Maltose 0.2% | 69.8% | 8.5 |
| Embodiment 3 | 20% | Soybean lecithin | Mannitol 2% | 76.5% | 8.1 |
| | | 1.5% | | | |
| Embodiment 4 | 15% | Yolk lecithin 0.8% | Glycerin | 80.6% | 7.5 |
| | | Soybean lecithin 2% | 1.6% | | |
| Embodiment 5 | 7% | Polyglycerol | Glucose 2% | 81% | 7.0 |
| | | palmitic diol 8% | Glycerin 2% | | |
| Embodiment 6 | 18% | Alginates 1% | Sorbitol 1% | 78% | 7.9 |
| | | Yolk lecithin 2% | | | |

### Embodiment 7

The contents Determination of 1,2-oleic-3-Palmitic Triglyceride and Glycerol Trioleate in Ganoderma lucidium Spore Oil Fat Emulsion

Determination with HPLC:

Chromatographic Conditions: With octadecyl silane as the bulking agent; Chromatographic column: Kromasil C18, 4.6 mm×250 mm, 5 um column; Temperature of Column: 30° C.; Mobile Phase: Acetonitrile: Isopropanol with a volume ratio of 40:60; Detecting with evaporative light-scattering detector; Flow rate: 0.5 ml/min. Number of theoretical plates shall be calculated pursuant to the peaks of 1,2-oleic-3-palmitic triglyceride or Glycerol Trioleate; both of which are higher than 2000;

The preparation of Control Solutions: Measure 1,2-oleic-3-palmitic triglyceride and Glycerol Trioleate controls of 2.98 mg and 2.67 mg respectively, place them into 25 ml of volumetric flasks, and then add into methanol to dissolve to volume, to formulate into solutions with 0.119 mg and 0.107 mg of content respectively in every 1 ml of solution, then the control solutions are obtained;

The preparation of testing solution: Accurately measure 1.025 g of Ganoderma lucidium spore oil Fat emulsion (Embodiment 1), then add into 0.2 g of Sodium Sulphate anhydorous, and then heat on water bath to crack the emulsion, then transferred into separating funnel, and then extract 3 times with diethyl ether, with a volume of 2.0 ml each time, and then merge the diethyl ether solution, to evaporate to dryness, and add into the mobile phase: dissolve in acetonitrile:isopropanol (40:60), then transfer into 100 ml volumetric flask, and dilute to volume until the testing solution is obtained;

Determination Result: Accurately measure 10 μl of control solution and testing solution respectively, and inject into High Performance Liquid Chromatograph, for determination and then carry out logarithms, and the result is obtained. Content of 1,2-oleic-3-palmitic triglyceride or Glycerol Trioleate in every 1 g of Emulsion are 4.21 mg and 3.43 mg respectively.

1,2-oleic-3-palmitic triglyceride content complies with 2 mg˜62.5 mg in every 1 g of Gonaderma lucidium spore oil Fat emulsion and/or Glycerol Trioleate content complies with 1.6 mg˜50.0 mg in every 1 g of Ganoderma lucidium spore oil Fat emulsion;

This indicates that the Fat emulsion Quality of Embodiment 1 complies with the requirement.

### Embodiment 8

The Contents Determination of 1,2-oleic-3-palmitic triglyceride and Glycerol Trioleate in Ganoderma lucidium Spore Oil Fat Emulsion

Determination with HPLC:

Chromatographic Conditions: With octadecyl silane as the bulking agent; Chromatographic column: Kromasil C18, 4.6 mm×250 mm, 5 um column; Temperature of Column: Room Temperature; Mobile Phase: Acetonitrile: Dicholromethane with a volume ratio of 59:41; detecting with evaporative light-scattering detector; Flow rate: 1.0 ml min. Number of theoretical plates shall be calculated pursuant to the peaks of 1,2-oleic-3-palmitic triglyceride or Glycerol Trioleate, both of which are higher than 2000;

The preparation of Control Solutions: Measure 1,2-oleic-3-palmitic triglyceride and Glycerol Trioleate controls of 2.98 mg and 2.67 mg respectively, place them into 25 ml of volumetric flasks, and then add into methanol to dissolve to volume, to formulate into solutions with 0.119 mg and 0.107 mg of content respectively in every 1 ml of solution, then the control solutions are obtained;

The preparation of testing solution: Accurately measure 302.45 mg of Ganoderma lucidium spore oil Fat emulsion (Embodiment 2), then place it into Soxhlet extractor, then add into 30 ml of diethyl ether, cold soaking overnight and then add into 50 ml of diethyl ether and extract for 8 hours on hot water bath, and then recover the diethyl ether as extraction till exhausted, then dissolve the residues in mobile phase: Acetonitrile: Dicholormethane (59:41), and transferred into 50 ml of the volumetric flask then diluted to volume until the testing solution is obtained;

Determination Result: Accurately measure 10 μl of control solution and testing solution respectively, and inject into High Performance Liquid Chromatograph, for determination and then carry out logarithms, and the result is obtained. Content of 1,2-oleic-3-palmitic triglyceride or Glycerol Trioleate in every 1 g of Ganoderma lucidium spore are 59.28 mg and 46.52 mg respectively.

1,2-oleic-3-palmitic triglyceride content complies with 2 mg˜62.5 mg in every 1 g of Gonaderma lucidium spore oil Fat emulsion and/or Glycerol Trioleate content complies with 1.6 mg˜50.0 mg in every 1 g of Ganoderma lucidium spore oil Fat emulsion;

This indicates that the Fat emulsion Quality of Embodiment 2 complies with the requirement.

### Embodiment 9

Content Determination of Ergosterol in Ganoderma lucidium Spore Oil Fat Emulsion

Determination with HPLC:

Chromatographic Conditions and Wavelength Selection: With octadecyl silane as the bulking agent; with methanol as the mobile phase; and the detection wavelength is 280 nm.The Number of Theoretical Plate is calculated pursuant to the peak of ergosterol, which shall not be less than 2000;

Preparation of Control Solution: measure the proper amount of ergosterol control, and then add into methanol to formulate the solution containing 0.08 mg of ergosterol in every 1 ml of solution, then the control solution is obtained;

The preparation of testing solution: Accurately measure 10 g of Ganoderma lucidium spore oil Fat emulsion (Embodiment 1), then add 2 g of Sodium Sulphate anhydorous, and heat on water bath to crack the emulsion, then transfer into separating funnel, and then extract 3 times with diethyl ether, with a volume of 20 ml each time, and then merge the diethyl ether solution, to evaporate to dryness, dissolve in 10 ml of petroleum ether, then add onto silicagel column after treatment (100˜200 mesh, 10 g, with a diameter of 15 mm), and finally elute with 120 ml of mixture of petroleum ether: Ethyl Acetate (90:10), and discard the eluent, and then elute with 120 ml of mixture of petroleum ether: Ethyl Acetate (80:20), and then collect the eluent, and evaporate to dryness, then dissolve the residue in methanol, and transfer into 10 ml of volumetric flask, dilute with mathanol to volume, agitate till homogeneity, then the testing solution is obtained.

Determination Result: Accurately measure 10 μl of control solution and testing solution respectively, and inject into High Performance Liquid Chromatograph, for determination and then carry out logarithms until the result is obtained. There is 0.6 mg of ergosterol in every 1 of emulsion.

The ergosterol content in every 1 g of Ganoderma lucidium spore oil Fat emulsion complies with the standard of 0.004 mg to 7.5 mg, indicating that the Fat emulsion in Embodiment 1 complies with the requirement.

### Embodiment 10

HPLC Standard Fingerprint of Ganoderma lucidium Spore Oil Fat Emulsion.

The preparation of reference substance solution: with Glycerol Trioleate as the reference substance, measure proper amount of Glycerol Trioleate control, and dilute with mobile phase, to formulate into solution containing 0.15 mg Glycerol Trioleate in every 1 ml of solution, as the reference substance solution.

The preparation of testing solution: Accurately measure 0.533 g of Ganoderma lucidium spore oil Fat emulsion (Embodiment 4), then add into 0.2 g of Sodium Sulphate anhydorous, and then heat on water bath to crack the emulsion, then transfer into separating funnel, and then extract 3 times with diethyl ether, with a volume of 2.0 ml each time, and then merge the diethyl ether solution, to evaporate to dryness, and add into the mobile phase: dissolve in acetonitrile: isopropanol (40:60), then transfer into 100 ml volumetric flask, and dilute to volume until the testing solution is obtained;

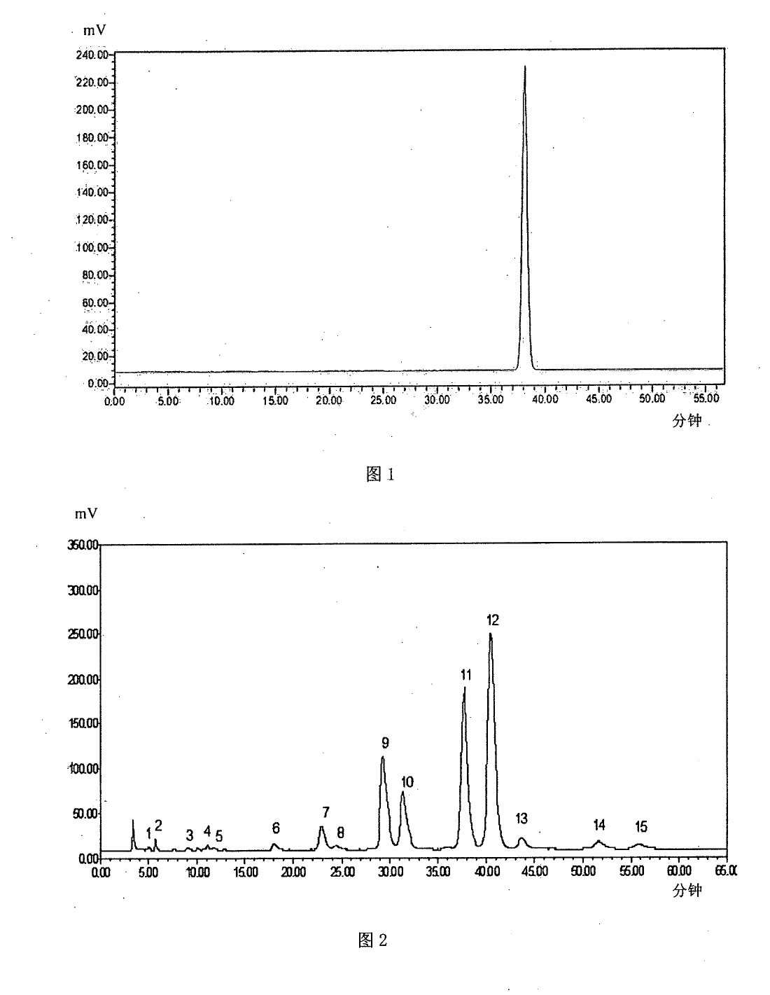

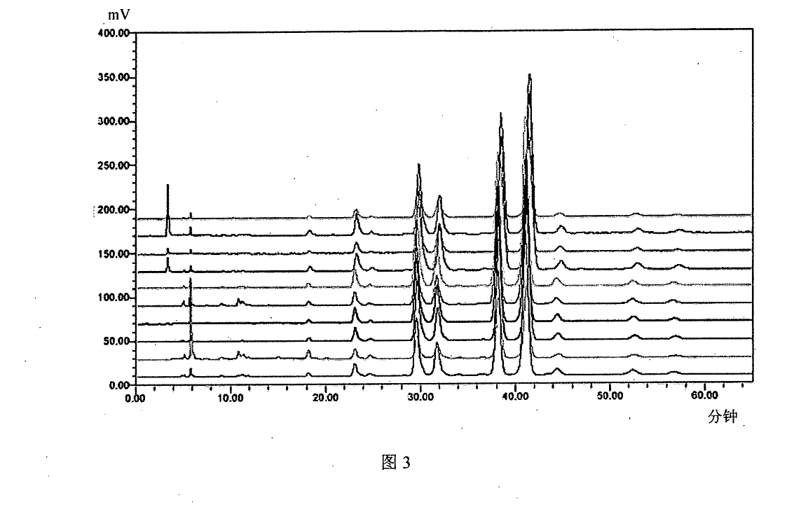

Measure 10 μl of reference substance solution and testing solution of Ganoderma lucidium spore oil Fat emulsion respectively for injection, determine with HPLC, to record the chromatogram of 60 minutes, which is referred to in FIGS. 1, 2 and 3. Calculate the relative retention time and relative peak area of other chromatogram peak with the relative retention time and relative peak area of Glycerol Trioleate Chromatogram as 1.

Determination of Common Peaks:

Through the fingerprint determination of 10 batches of Ganoderma lucidium spore oil Fat emulsion, they can be compared with HPLC Chromatogram, to determine the common characteristic peaks, and acquire the HPLC standard fingerprint of Ganoderma lucidium spore oil Fat emulsion constituted of their common characteristic peaks. This standard fingerprint has 15 characteristic peaks, with the RSD of the relative retention time of various peaks all smaller than 2%. Among which, Peak 1 has an average RT of 0.133, with RSD of 0.31%, relative peak area range between 0.10% and 2.40%; Peak 2 has an average RT of 0.152, with RSD of 0.32%, relative peak area between 0.31% and 17.51%; peak 3 has an average RT of 0.239, with RSD of 1.18%, relative peak area between 0.14% and 1.20%; Peak 4 has an average RT of 0.285, with RSD of 0.11%, relative peak area between 0.10% and 2.16%; Peak 5 has an average RT of 0.296, with RSD of 0.71%, relative peak area between 0.19% and 2.05%; Peak 6 has an average RT of 0.479, with RSD of 0.12%, relative peak area between 0.47 and 3.56%; Peak 7 has an average RT of 0.608, with RSD of 0.11%, relative peak area between 1.70% and 4.06%; Peak 8 has an average RT of 0.648, with RSD of 0.11%, relative peak area between 0.34% and 1.80%; Peak 9 has an average RT of 0.778, with RSD of 0.12%, relative peak area between 9.54% and 15.36%; Peak 10 has an average RT of 0.832, with RSD of 0.10%, relative peak area between 5.76% and 9.43%; Peak 11, i.e the reference peak of Glycerol Trioleate has an average RT of 1.000, relative peak area between 22.29% and 27.80%; Peak 12 has an average RT of 1.075, with RSD of 0.10%, relative peak area between 26.82% and 37.76%; Peak 13 has an average RT of 1.158, with RSD of 0.21%, relative peak area between 1.31% and 2.03%; Peak 14 has an average RT of 1.370, with RSD of 0.13%, relative peak area between 1.22% and 1.72%; Peak 15 has an average RT of 1.479, with RSD of 0.15%, relative peak area between 0.54% and 1.03%.

### Embodiment 11

Stability Test of Ganoderma lucidium Spore Oil Fat Emulsion:

Carry out 6 months of acceleration test for samples in Embodiment 4, observation conditions: Placed at 37° C., with a relative humidity of 90%. (Endotoxin Examination and Sterile Inspection Method shall be carried out pursuant to the relevant provisions in Chinese Pharmacopoeia 2005 Edition), with the experiment results demonstrated in Table 2:

TABLE 2
Observation Results of Acceleration Test lasting for 6 months

| Embodiment 4 | 0 month | 1 month | 2 month | 3 month | 6 month |
|---|---|---|---|---|---|
| pH value | 7.5 | 7.4 | 7.5 | 7.3 | 7.4 |
| Average Particle | 280 nm | 290 nm | 285 nm | 298 nm | 290 nm |
| Diameter | | | | | |
| Peroxide Value | 1.22 meq/kg | 1.13 meq/kg | 1.25 meq/kg | 1.28 meq/kg | 1.26 meq/kg |
| 1,2-oleic-3-palmitic | 31.8 mg/g | 31.1 mg/g | 31.6 mg/g | 31.7 mg/g | 31.2 mg/g |
| triglyceride | | | | | |
| Content of | 22.4 mg/g | 21.9 mg/g | 21.7 mg/g | 22.1 mg/g | 21.9 mg/g |
| Glycerol Trioleate | | | | | |
| Content of | 1.53 m/g | 1.52 m/g | 1.50 m/g | 1.51 m/g | 1.49 m/g |
| Ergosterol | | | | | |
| Endotoxin Test | Comply | Comply | Comply | Comply | Comply |
| | with the | with the | with the | with the | with the |
| | Provisions | Provisions | Provisions | Provisions | Provisions |
| Sterile Test | Comply | Comply | Comply | Comply | Comply |
| | with the | with the | with the | with the | with the |
| | Provisions | Provisions | Provisions | Provisions | Provisions |

Experiment result demonstrates that the experiment samples are basically stable during the Acceleration Inspection Period.

### Embodiment 12

Security Test of Ganoderma lucidium Spore Oil Fat Emulsion:

1. Active systemic anaphylaxis (ASA) in Guinea Pigs: There are 24 Hartley

Guinea pigs, randomized into 4 groups pursuant to gender and weight, and there are 3 males and 3 females in each group. They are divided into negative control group, positive control group, low dosage group for testing substance, and high dosage group for testing substance. Intraperitoneal injection is administered, with a dosage volume of 0.5 mL/animal, once every other day, for 5 times successively. All groups of animals are activated at day 12 after the final Intraperitoneal injection, with a dosage volume of 2.0 mL/animal, and observe the reaction of animals, and the allergic reaction incidence and their extent to determine the Active systemic anaphylaxis. Results: In the negative control group, there occurs 1 case of urination phenomenon in Guinea pigs (determined to be physiological urination), and there are no abnormalities in the remaining 5 guinea pigs, therefore the incidence of allergic reaction in negative control group is 0; guinea pigs in the positive control group are all dead, with 100% incidence of allergic reaction; in low dosage group and high dosage group, there is 1 case of guinea pig urination or defecation respectively (it is determined to be physiological urination or defecation), and there are no abnormality in the remaining guinea pigs, therefore the allergic reaction incidence in both the low dosage group and high dosage group are both 0; suggesting that under the current experiment conditions, the low dosage group and high dosage group of Ganoderma lucidium spore oil fat emulsion (62.5, 125.0 mg/kg·bw) cause no significant Active systemic anaphylaxis (ASA) reaction in Hartley guinea pigs.

2. Passive cutaneous anaphylaxis in Rats (PCA): There are 24 SPF Grade SD Rats, randomized into 4 groups pursuant to gender and weight, and there are 3 males and 3 females in each group. They are divided into negative control group, positive control group, low dosage group and high dosage group for testing substance (respectively 125 and 250 mg/kg·bw, correspond respectively to 0.14 and 0.28 time of clinical application dosage of 10 grams, calculated pursuant to Equivalent Dose.) Intraperitoneal injection administered, once every other day, for 5 times successively. On day 10 after the last sensitization, prepare the antiserum. Take 24 SPF Grade SD Rats, dividing into the same groups as the method for antibody preparation mentioned above, and all groups of animals are intracutaneous injected with 0.1 mL of antiserum of all corresponding groups, activate 48 hours after passive sensitization, with the results demonstrating that there are no blue spot on the inner side of dorsal skin for negative control group and low dosage as well as high dosage group, for positive control group, evident blue spots were detected. It is suggested that under the present experiment conditions, there detects no evident allergic reaction in SD Rats injected with Ganoderma lucidium spore oil fat emulsion for passive cutaneous anaphylaxis test.

3. Stimulation on blood vessels of rabbits: Take 4 healthy New Zealand Rabbits, apply the self-contrast method between the left and right ear of the same animal, the left ear is injected with the testing drug (100 mg/mL, equivalent to the concentration used in clinical intravenous infusion), and the right ear is injected with 0.9% NaCl normal saline water of the same volume, once daily, for 7 successive days. 48 hours after the last administration of the drug, gross examine two animals, and the remaining animals are gross examined at day 14 after last administration. Naked eye and Microscopic Examination: there was no pathological changes in veins on the rabbit's ear detected on both the administered drug side and the control side as well as the surrounding tissues. It is suggested that under the present experiment conditions, there was no evident stimulation reaction detected in rabbits injected with Ganoderma lucidium spore oil fat emulsion in veins on the rabbit's ears and the surrounding tissues.

4. Rabbit Muscular Stimulation Test: Take 4 healthy New Zealand Rabbits, apply the self-contrast method between the left and right side muscle of quadriceps femoris of the same animal, the left side is injected with testing drug (100 mg/mL, equivalent to the concentration used in clinical intravenous infusion), and the right side is injected with 0.9% NaCl normal saline water of the same volume, administered with a volume of 1.0 mL/side, dosed once. 48 hours after the last administration of drug, gross examine two animals, the remaining animals are gross examined at day 14 after administration. There was no evident abnormality detected in injected muscle on both sides in four rabbits observed by the naked eye. Microscopic Examination Results: 48 hours after dosing, gross examine one animal and on day 14, gross examine two animals, with muscle fibres aligned evenly in the injection sites on both sides, there were no abnormal changes detected. For the remaining rabbit, 48 hours after the last dosage, in local muscle tissues on the right side, there was slight degeneration of small fibrous nidus detected, with a large amount of inflammatory cell infiltration in the muscle fiber stromal of the nidus, in the muscle tissue on the left side, there was flake degeneration detected, necrosis even disappearance in muscle tissues, and there was a large amount of inflammatory cell infiltration in nidus and muscle fibers, there was a small amount of Red Blood Cells. With the right side as the negative control, there was slight mechanical stimulation, in combination with naked eye observation, the stimulation in local tissues on the left side is possibly mechanical stimulation. It is suggested that under the present experiment conditions, there was no evident stimulation reaction in rabbits injected with Ganoderma lucidium spore oil fat emulsion in quadriceps femoris muscle.

5. Hemolysis Test in vitro: add a different amount of Ganoderma lucidium spore oil fat emulsion into each drug tube suspended with 2% Red Blood Cells (100 mg/mL, about equivalent to the clinical concentration in intravenous infusion), there was no hemolyisis or Red Blood Cell agglomeration in each drug tube with Ganoderma lucidium spore oil fat emulsion within 3 hours. It is suggested that under the present experiment conditions, for the three batches of Ganoderma lucidium spore oil fat emulsion, hemolysis test in vitro had negative results.

### Embodiment 13

Acute Beagle Dog Toxicity Test of Ganoderma lucidium Spore Oil:

The experiment establishes 5 dosage groups with the dosages of 4, 6, 8, 12, 16 g/kg·bw (corresponding to 24, 36, 48, 72, 96 times of clinical dosage in human), there is one Beagle Dog in each dosage group, intravenously administered once. Experiment results indicate: After single dosage to a Beagle dog, there was no evident abnormality in electrocardiogram, hematology and urine examination, however there are some changes in hematological chemistry, the higher the dosage, the longer the lasting time; when the dosage reaches 12, 16 g/kg·bw, there were such symptoms as being quiet, reduction in activity, and decrease in appetite, etc in animals. Single intravenous infusion of Ganoderma lucidium spore oil fat emulsion to a Beagle dog has an non-toxic effect dose of 4 g/kg·bw, with a tolerance higher than 16 g/kg·bw.

### Embodiment 14

Experiment Treatment Effect of Ganoderma lucidium Spore Oil Fat Emulsion i.v. Dosage Against Mice H₂₂ Hepatoma.

Select H₂₂ tumors with excellent growth at day 7 to day 11, innoculate intracutaneously under the right armpits of the mice, about 4.5˜5×10⁶ cells/animal, 24 hours after innoculation, these mice are randomly separated into different cages, and intravenously administered. During dosing, the body weight of the animals are measured daily, who are sacrificed on day 8, with the tumor weight measured, to calculate the average tumor weight in each group, as well as the inhibition rate of tumors and carry out t Test.

TABLE 3
Tumor growth inhibition effect of Ganoderma lucidium spore oil fat
emulsion i.v. dosage against mice H₂₂ Hepatoma ( X̅ ± SD).

| | | Number of | | | | Tumor | |
|---|---|---|---|---|---|---|---|
| | Dosage | Animals | | Body Weight (g) | | Weight | Inhibition |
| Group | mg/kg | d1 | d8 | d1 | d8 | (g) | Rate (%) |
| Blank | | 10 | 10 | 19.9 ± 1.0 | 26.5 ± 3.2 | 1.66 ± 0.33 | |
| Emulsion | | | | | | | |
| CTX | 30 | 10 | 10 | 18.9 ± 1.2 | 24.5 ± 3.1 | 0.69 ± 0.21** | 58.4 |
| Ganoderma | 1.0 g/kg | 10 | 10 | 19.0 ± 0.7 | 26.4 ± 3.3 | 0.92 ± 0.33** | 44.6 |
| lucidium spore | | | | | | | |
| oil fat | | | | | | | |
| emulsion for | | | | | | | |
| injection | | | | | | | |
| Ganoderma | 0.5 g/kg | 10 | 10 | 19.6 ± 0.9 | 27.8 ± 2.8 | 1.05 ± 0.29** | 36.7 |
| lucidium spore | | | | | | | |
| oil fat | | | | | | | |
| emulsion for | | | | | | | |
| injection | | | | | | | |
| Ganoderma | 0.25 g/kg | 10 | 10 | 19.7 ± 1.2 | 29.1 ± 2.8 | 1.31 ± 0.52 | 21.1 |
| lucidium spore | | | | | | | |
| oil fat | | | | | | | |
| emulsion for | | | | | | | |
| injection | | | | | | | |

d1, d8: Day 1 and Day 8 after innoculation
**P < 0.05, Comparing with Blank Emulsion.

Data from Table 3 indicates, Ganoderma lucidium spore oil fat emulsion could inhibit the tumor growth of transplant mice tumor H₂₂ (Hepatoma).

### Embodiment 15

Experiment Treatment Effect of Ganoderma lucidium Spore Oil Fat Emulsion i.v. Dosage Against Human Gastric Adenocarcinoma SGC-7901 Transplanted in Nude Mice.

Take nude mice transplanted with tumor tissues of Human gastric adenocarcinoma SGC-7901 in vigorous growth period, shred into pieces of about 1.5 mm³, under sterile conditions, innoculate under the skin of the right armpits of nude mice. Transplanted tumor diameters in nude mice are measured with vernier caliper, till the tumors grow to 100 to 300 mm³, and randomized into groups. Apply the method to measure the tumor diameters, dynamically observe the antitumor effects of the testing substance. The tumor diameters are measured 3 times every week, measuring the weight of mice at each time. Experiment group is

Intravenously Administered, once every other day; positive control medicine is TAX, 10 mg/kg, once every other day; and the negative control group is the blank emulsion with equivalent volume. Evaluate the growth inhibition effects of the drug against tumor cells with relative tumor inhibition rate T/C (%). The Testing Results are:

TABLE 4
Experiment treatment effect of Ganoderma lucidium spore oil fat
emulsion i.v. dosage against human gastric adenocarcinoma SGC-7901
transplanted in nude mice ( X̅ ± SD).

| | | Number of | | | | | | | | |
|---|---|---|---|---|---|---|---|---|---|---|
| | Dosage | Animals | | Body Weight (g) | | TV (x ± SD) | | RTV | T/C | P |
| Group | mg/kg | Beginning | End | Beginning | End | d1₂ | d₃₃ | ( X̅ ± SD) | (%) | value |
| Blank | | 12 | 12 | 18.4 ± 1.3 | 21.8 ± 1.0 | 157 ± 131 | 3055 ± 2804 | 19.01 ± 5.95 | | |
| Emulsion | | | | | | | | | | |
| TAX | 10 | 6 | 6 | 18.0 ± 1.9 | 19.1 ± 0.8 | 144 ± 103 | 1021 ± 537 | 8.71 ± 3.55 | 45.8 | <0.05 |
| Ganoderma | 1 g/kg | 6 | 6 | 19.0 ± 1.0 | 24.0 ± 1.6 | 143 ± 56 | 1508 ± 415 | 11.02 ± 2.37 | 58.0 | <0.05 |
| lucidium | | | | | | | | | | |
| spore oil | | | | | | | | | | |
| fat | | | | | | | | | | |
| emulsion | | | | | | | | | | |
| for | | | | | | | | | | |
| injection | | | | | | | | | | |
| Ganoderma | 0.5 g/kg | 6 | 6 | 18.6 ± 1.1 | 22.9 ± 1.6 | 156 ± 92 | 2013 ± 863 | 14.04 ± 3.24 | 73.9 | |
| lucidium | | | | | | | | | | |
| spore oil | | | | | | | | | | |
| fat | | | | | | | | | | |
| emulsion | | | | | | | | | | |
| for | | | | | | | | | | |
| injection | | | | | | | | | | |
| Ganoderma | 0.25 g/kg | 6 | 6 | 18.2 ± 1.4 | 23.2 ± 1.2 | 142 ± 26 | 2420 ± 630 | 16.98 ± 2.43 | 89.3 | |
| lucidium | | | | | | | | | | |
| spore oil | | | | | | | | | | |
| fat | | | | | | | | | | |
| emulsion | | | | | | | | | | |
| for | | | | | | | | | | |
| injection | | | | | | | | | | |

d₉: is the time for administration after dividing into different cages.

Data from Table 4 indicate that Ganoderma lucidium spore oil fat emulsion i.v. dosage had inhibition effects against human gastric adenocarcinoma SGC-7901 transplanted in nude mice.

### Embodiment 16

Experiment Treatment Effect of Ganoderma lucidium Spore Oil Fat Emulsion i.v. Dosage Influencing the Immunity Function of Specific Cells in H₂₂ Tumor Bearing Mice

Take 50 ICR mice, inoculate with H₂₂ solid tumor pursuant to the research method of transplantable tumor (take the tumor mass under sterile operation, then weigh, grinding with Glass tissue homogenizer, after homogeneity, put into sterile containers, then dilute with normal saline into cell suspension of 1:3, for each mouse, inoculate intracutaneously under the right armpit for 0.2 ml). On the second day, they are randomized into 5 groups, with 10 mice in each group, with half being males and half females, which are divided into: Intravenous Control Group (20 mg/kg, Blank Group), High, Medium, Low Doasge Groups injected with Ganoderma lucidium spore oil fat emulsion (1, 0.5, 0.25 g/kg) iv administered, Thymosin a1 injection group (0.42 mg/kg) Sc administered; apart from that Thymosin a1 is administered once every other day, other groups are administered once daily, with 7 administration. On day 1 after dosage, hair is removed from the abdomen of each mouse, with an area of 3×3 cm². On day 2 after dosage, smear 1% of DNFB evenly on the abdomen of mice (DNFB is a kind of haptene, which after combination with cutaneous proteins could form holoantigen, that stimulate the T Lymphocytes to multiply into sensitized lymphocytes). Half an hour after the last dosage, smear the 1% of DNFB on the right ear of the mice to attack, with the occurrence of delayed allergy (edema) in local areas, 24 hours after the attack, execute the mice by breaking the cervical vertebra, then cut off the left and the right ears, puncture a piece of the ear of 8mm diameter with puncher, then weigh, calculate the weight difference between the left and the right ear as the degree of swelling, and then calculate the swelling inhibition rate, to compare the differences between different groups.

TABLE 5
Delayed Hypersensitivity of Ganoderma lucidium spore oil fat
emulsion i.v. dosage on tumor bearing mice of H₂₂ Hepatoma ( X̅ ± SD, n = 10).

| | | Body | Body | | |
|---|---|---|---|---|---|
| | | Weight | Weight | Swelling | |
| | Dosage | before | after | Degree | Swelling |
| Group | (mg/kg) | Dosage | Dosage | (mg) | Rate (%) |
| Blank Emulsion | | 19.50 ± 1.35 | 25.60 ± 2.12 | 9.16 ± 3.68 | 62.06 ± 24.11 |
| Control | | | | | |
| Ganoderma | 1000 | 19.40 ± 1.35 | 25.50 ± 2.12 | 12.73 ± 3.70* | 88.52 ± 28.94* |
| lucidium spore | 500 | 19.80 ± 1.69 | 25.30 ± 2.11 | 12.84 ± 3.79* | 76.53 ± 15.19 |
| oil fat emulsion | 250 | 19.90 ± 1.60 | 25.10 ± 2.13 | 11.87 ± 2.30 | 72.74 ± 11.33 |
| for injection | | | | | |
| Thymosin a1 | 0.42 | 19.30 ± 1.34 | 25.10 ± 2.18 | 13.50 ± 3.44* | 89.12 ± 19.76* |

*P < 0.05,
**P < 0.01 Compared with Blank Emulsion Control Group with H₂₂ Tumor

Data from Table 5 indicate, High dosage and Medium dosage group of Ganoderma lucidium spore oil fat emulsion can significantly improve the swelling degree and swelling rate (P<0.05). Namely Ganoderma lucidium spore oil fat emulsion can strengthen the immunity function of specific cells in H₂₂ tumor bearing mice.

### Embodiment 17

Experiment Treatment Effect of Ganoderma lucidium Spore Oil Fat Emulsion i.v. Dosage Influencing the Phagocytic Activity of Reticuloendothelial System (RES) in H₂₂ Tumor Bearing Mice.

Experiment Method: Take 50 ICR mice, inoculate with H₂₂ solid tumor, randomize into 5 groups pursuant to body weight, with 10 animals in each group, half of which are females and half of which are males. In the experiment there are: Intravenous control group (20 ml/kg, Blank Emulsion), High dosage, Medium dosage and Low dosage group of Ganoderma lucidium spore oil fat emulsion for injection (1, 0.5, 0.25 g/kg) iv administered, Positive control drug Thymosin a1 injection group (0.42 mg/kg) Sc administered. Apart from that Thymosin a1 is administered once every other day, other groups are administered once daily, with a total dosage of 11 times. 24 hours after iv, ig administration for the last time, inject into the caudal vein the indian ink (diluted as 1:3) 0.1 ml/10 g, use quantitative blood collection tube to collect 20 μl of blood from post orbit venous plexus at 1 minute and 5 minute after ink injection, then pipette into 2 ml of 0.1% Na₂CO₃, and then carry out colormetric assay at 680 nm, then the mice are sacrificed at 5 minute after blood collection, and then weigh the liver, spleen, thymus gland, calculate the clearance index K and phagocytic coefficient α value as well as the liver coefficient and spleen coefficient pursuant to the following equation. All the data obtained are statistically treated (t Test).

TABLE 6
Influence of Ganoderma lucidium spore oil fat emulsion i.v. dosage on
the phagocytic activity of reticuloendothelial system (RES) in H₂₂ tumor bearing
mice ( X̅ ± SD, n = 10).

| | | | | | | Thymus |
|---|---|---|---|---|---|---|
| | | | | Liver | Spleen | Gland |
| | Dosage | Clearance | Phagocytic | Coefficient | Coefficient | Coefficient |
| Group | (mg/kg) | Index K | Coefficient α | (g/100 g) | (g/100 g) | (g/100 g) |
| Blank | | 0.05 ± 0.02 | 4.98 ± 0.76 | 6.52 ± 0.98 | 0.85 ± 0.14 | 0.24 ± 0.04 |
| Emulsion | | | | | | |
| Control | | | | | | |
| Ganoderma | 1000 | 0.10 ± 0.03** | 5.91 ± 1.16* | 7.03 ± 1.40 | 1.00 ± 1.32 | 0.33 ± 0.12* |
| lucidium | 500 | 0.11 ± 0.02** | 5.38 ± 0.71 | 7.76 ± 1.56* | 1.18 ± 0.46* | 0.32 ± 0.10* |
| spore oil | 250 | 0.10 ± 0.02** | 5.51 ± 1.13 | 7.03 ± 1.52 | 1.55 ± 0.58** | 0.27 ± 0.08 |
| fat | | | | | | |
| emulsion | | | | | | |
| for | | | | | | |
| injection | | | | | | |
| Positive | 0.42 | 0.09 ± 0.02** | 5.71 ± 0.76* | 6.91 ± 1.08 | 1.16 ± 0.44* | 0.33 ± 0.11* |
| Control: | | | | | | |
| Thymosin | | | | | | |
| a1 | | | | | | |

*P < 0.05,
**P < 0.01 Compared with Blank Emulsion Control Group with H₂₂ Tumor

Data from Table 6 indicate, Ganoderma lucidium spore oil fat emulsion could significantly improve the clearance index K, phagocytic coefficient a and spleen coefficient in H₂₂ tumor bearing mice, and strengthen the phagocytic function of reticuloendothelial system (RES) in H₂₂ tumor bearing mice.

### Embodiment 18

Toxicity Reduction Effect of Ganoderma lucidium Spore Oil Fat Emulsion i.v. Dosage for Chemotherapy with Cyclophosphamide (Cy) in H₂₂ Tumor Bearing Mice

Experiment Method: Take 60 ICR mice, inoculate with H₂₂ solid tumor, weigh 24 hours after inoculation, then randomize into 6 groups, with 10 animals in each group, half of which are females and half of which are males. In the experiment there are: Blank control group, Intravenous control group (20 ml/kg, Blank Emulsion), High dosage, Medium dosage and Low dosage group of Ganoderma lucidium spore oil fat emulsion for injection (1, 0.5, 0.25 g/kg) iv administered, Positive control drug Thymosin a1 injection group (0.42 mg/kg) Sc administered. Apart from that Thymosin a1 are dosed once every other day, the other groups are administered once daily, with a total dose of 7 times, on day 4 and day 5 of administration, apart from blank control, the other groups begin to be treated with ip.Cy (100 mg/kg), for two successive day, and the mice are sacrificed 24 hours after termination of dosage, weigh and collect blood from the orbit veins before sacrificing, microscopic examine the total number of peripheral blood leukocytes, at the same time dissect one complete femur, then determine the number of nucleated cells in the bone marrow, and remove the thymus gland and spleen for weighing, and calculate the coefficients of thymus glands and spleen organs, and carry out statistical treatment (t Test).

Experiment Results: Results indicate, compared with blank control group of H₂₂ tumor bearing mice, Peripheral blood leukocytes, and the number of nucleated cells in bone marrow as well as thymus gland and spleen coefficients in Blank Emulsion+Cy model (100 mg/kg, ip) decline significantly (P<0.01). Comparing with cyclophosphamide (Cy)model group, High dosage group of Ganoderma lucidium spore oil fat emulsion for injection and Thymosin a1 injection group could both significantly withstand the decline in mice peripheral blood leukocytes, the number of nucleated cells in bone marrow, thymus gland coefficient and spleen coefficient (P<0.05, P<0.01) caused by Cy in H₂₂ tumor bearing mice. The results are demonstrated in Table 7.

TABLE 7
Toxicity reducing effect of Ganoderma lucidium spore oil fat emulsion
i.v. dosage for chemotherapy in H₂₂ tumor bearing mice ( X̅ ± SD, n = 10)

| | | | | | | | The number |
|---|---|---|---|---|---|---|---|
| | | Body Weight | | | Thymus | | of nucleated |
| | | (g) | | Spleen | Gland | | cells in bone |
| | Dosage | Before | After | Coefficient | Coefficient | WBC | marrow |
| Group | (mg/kg) | Dosage | Dosage | (g/100 g) | (g/100 g) | (10⁹/L) | (10⁵/piece) |
| Blank | | 19.80 ± 1.75 | 24.00 ± 2.11 | 1.29 ± 0.47** | 0.20 ± 0.06** | 5.61 ± 1.14** | 191.03 ± 30.65** |
| Control | | | | | | | |
| Blank | 100 | 19.90 ± 1.60 | 22.70 ± 2.16 | 0.48 ± 0.13 | 0.08 ± 0.04 | 1.63 ± 0.40 | 60.23 ± 11.18 |
| Emulsion + | | | | | | | |
| Cy | | | | | | | |
| Ganoderma | 1000 + 100 | 19.60 ± 1.43 | 22.30 ± 2.11 | 0.60 ± 0.12* | 0.12 ± 0.04* | 2.02 ± 0.43* | 73.15 ± 14.93* |
| lucidium | 500 + 100 | 20.00 ± 1.83 | 23.00 ± 2.16 | 0.57 ± 0.21 | 0.10 ± 0.03 | 1.73 ± 0.61 | 68.79 ± 12.51 |
| spore oil fat | 250 + 100 | 19.70 ± 1.57 | 23.00 ± 2.26 | 0.58 ± 0.16 | 0.11 ± 0.04 | 1.91 ± 0.65 | 61.48 ± 12.50 |
| emulsion for | | | | | | | |
| injection + | | | | | | | |
| Cy | | | | | | | |
| Thymosin | 0.42 + 100 | 19.70 ± 1.57 | 23.60 ± 2.27 | 0.64 ± 0.21* | 0.12 ± 0.04* | 2.03 ± 0.44* | 73.31 ± 15.99* |
| a1 + Cy | | | | | | | |

*P < 0.05,
**P < 0.01 Compared with Blank Emulsion + Cy group.

Data from Table 7 indicate that Ganoderma lucidium spore oil fat emulsion i.v. dosage has toxicity reducing effects for chemotherapy with cyclophosphamide (Cy) in H₂₂ tumor bearing mice.

### Embodiment 19

Toxicity reducing Effect of Ganoderma lucidium Spore Oil Fat Emulsion i.v. Dosage for Chemotherapy (Cy) in Normal Mice

Experiment Method: Take 60 ICR mice, and randomize into 6 groups pursuant to body weight, with 10 animals in each group, half of which are females and half of which are males. In the experiment there are: Blank control group, Intravenous control group (20 ml/kg, Blank Emulsion), High dosage, Medium dosage and Low dosage groups of Ganoderma lucidium spore oil fat emulsion for injection (1, 0.5, 0.25 g/kg) iv administered, Positive control drug Thymosin a1 injection group (0.42 mg/kg) Sc administered. Apart from that Thymosin a1 is administered once every other day, other groups are administered once daily, with a total dosage of 7 times. On day 4 and day 5 of the administration, apart from blank control, the other groups begin to be treated with ip.Cy (100 mg/kg), for two successive days, and the mice are sacrificed 24 hours after termination of dosage, weigh and collect blood from the orbit veins before sacrificing, microscopically examine the total number of peripheral blood leukocytes, at the same time dissect one complete femur, then determine the number of nucleated cells in the bone marrow, and remove the thymus gland and spleen for weighing, and calculate the coefficients of thymus glands and spleen organs, and carry out statistical treatment (t Test).

Experiment Results: Results indicate, compared with blank control group, Peripheral blood leukocytes, and the number of nucleated cells in bone marrow as well as thymus gland and spleen coefficients in Blank Emulsion+Cy model (100 mg/kg, ip) decline significantly (P<0.01). Compared with blank emulsion+Cy group, High dosage group of Ganoderma lucidium spore oil fat emulsion for injection and Thymosin a1 injection group could both significantly withstand the decline in mice peripheral blood leukocytes, the number of nucleated cells in bone marrow, thymus gland coefficient and spleen coefficient (P<0.05) in normal mice caused by Cy, and Medium dosage group of Ganoderma lucidium spore oil fat emulsion for injection could significantly withstand the decline of the number of nucleated cells in bone marrow, spleen coefficient (P<0.05) in normal mice caused by Cy, with the results demonstrated in Table 8.

TABLE 8
Toxicity reducing effect of Ganoderma lucidium spore oil fat emulsion
i.v. dosage for chemotherapy in normal mice ( X̅ ± SD, n = 10).

| | | | | | Thymus | | The number of |
|---|---|---|---|---|---|---|---|
| | | Body Weight (g) | | Spleen | Gland | | nucleated cells in |
| | Dosage | Before | After | Coefficient | Coefficient | WBC | bone marrow |
| Group | (mg/kg) | Dosage | Dosage | (g/100 g) | (g/100 g) | (10⁹/L) | (10⁵/piece) |
| Blank | | 19.40 ± 1.07 | 25.20 ± 2.15 | 0.55 ± 0.19** | 0.32 ± 0.14** | 10.67 ± 3.03** | 200.18 ± 21.17** |
| Control | | | | | | | |
| Blank | 100 | 19.80 ± 1.32 | 23.20 ± 2.20 | 0.28 ± 0.06 | 0.12 ± 0.04 | 1.60 ± 0.50 | 62.14 ± 14.57 |
| Emulsion + | | | | | | | |
| Cy | | | | | | | |
| Ganoderma | 1000 + 100 | 19.10 ± 1.20 | 23.00 ± 2.11 | 0.34 ± 0.06* | 0.12 ± 0.06 | 1.98 ± 0.26* | 77.29 ± 14.41* |
| lucidium | 500 + 100 | 19.90 ± 1.37 | 23.30 ± 2.11 | 0.35 ± 0.08* | 0.13 ± 0.07 | 1.75 ± 0.55 | 74.89 ± 11.44* |
| spore oil | 250 + 100 | 19.30 ± 1.16 | 23.10 ± 2.13 | 0.35 ± 0.08* | 0.13 ± 0.05 | 1.61 ± 0.46 | 65.73 ± 19.41 |
| fat | | | | | | | |
| emulsion | | | | | | | |
| for | | | | | | | |
| injection + | | | | | | | |
| Cy | | | | | | | |
| Thymosin | 0.42 + 100 | 19.60 ± 1.26 | 22.90 ± 2.18 | 0.36 ± 0.10* | 0.18 ± 0.03** | 2.04 ± 0.41* | 75.43 ± 13.32* |
| a1 + Cy | | | | | | | |

*P < 0.05,
**P < 0.01 Comparing with Blank Emulsion + Cy group.

Data from Table 8 indicate that Ganoderma lucidium spore oil fat emulsion i.v. dosage has toxicity reducing effects for chemotherapy with cyclophosphamide (Cy) in normal mice.

### Embodiment 20

Toxicity Reducing Effect of Ganoderma lucidium Spore Oil Fat Emulsion i.v. Dosage for Chemotherapy with ⁶⁰Co in H₂₂ Tumor Bearing Mice

Experiment Method: Take 60 ICR mice, innoculate with H₂₂ solid tumor pursuant to the research method for transplantable tumors, 24 hours after innoculation, apart from the 10 animals in blank control, the other 50 mice receive ⁶⁰Co radiation, with a dosage of 500 rad; after radiation with ⁶⁰Co, randomize the animals into 5 groups pursuant to body weight, with 10 animals in each group, half of which are females and half of which are males. In the experiment there are: Blank control group, Intravenous control group (20 ml/kg, Blank Emulsion), High dosage, Medium dosage and Low dosage group of Ganoderma lucidium spore oil fat emulsion for injection (1, 0.5, 0.25 g/kg) iv administered, Positive control drug Thymosin a1 injection group (0.42 mg/kg) Sc administered. Apart from that Thymosin a1 are dosed once every other day, the other groups are administered once daily, with a total dose of 7 times, and the mice are sacrificed 24 hours after termination of dosage, weigh and collect blood from the orbit veins before sacrificing, microscopically examine the total number of peripheral blood leukocytes, at the same time remove spleen, thymus gland and one complete femur, then determine the number of nucleated cells in bone marrow, and weigh the thymus gland and spleen, and calculate the coefficients of thymus glands and spleen organs, and carry out statistical treatment (t Test).

Experiment Results: Results indicate, compared with blank control group of H₂₂ tumor bearing mice, Peripheral blood leukocytes, and the number of nucleated cells in bone marrow as well as the thymus gland and spleen coefficients in Blank Emulsion+⁶⁰Co model (500 rad) decline significantly (P<0.01). Compared with ⁶⁰Co model group, Thymosin a1 injection group could significantly withstand the decline in mice peripheral blood leukocytes, the number of nucleated cells in bone marrow, thymus gland coefficient and spleen coefficient (P<0.05) in H₂₂ tumor bearing mice caused by ⁶⁰Co, and High dosage group of Ganoderma lucidium spore oil fat emulsion for i.v. injection could significantly withstand the decline of the number of peripheral blood leukocytes, the number of nucleated cells in bone marrow, and spleen coefficient (P<0.05) in H₂₂ tumor bearing mice caused by ⁶⁰Co, and Medium dosage group of Ganoderma lucidium spore oil fat emulsion for i.v. injection could significantly withstand the decline of the number of peripheral blood leukocytes, (P<0.05) in H₂₂ tumor bearing mice caused by ⁶⁰Co. The results are demonstrated in Table 9.

TABLE 9
Toxicity reducing effect of Ganoderma lucidium spore oil fat emulsion
i.v. dosage for radiotherapy of ⁶⁰Co in H₂₂ tumor bearing mice ( X̅ ± SD, n = 10).

| | | | | | | | The number |
|---|---|---|---|---|---|---|---|
| | | Body Weight | | | Thymus | | of nucleated |
| | | (g) | | Spleen | Gland | | cells in bone |
| | Dosage | Before | After | Coefficient | Coefficient | WBC | marrow |
| Group | (mg/kg) | Dosage | Dosage | (g/100 g) | (g/100 g) | (10⁹/L) | (10⁵/piece) |
| Blank Control | | 19.40 ± 1.26 | 24.50 ± 2.37 | 0.62 ± 0.20** | 0.27 ± 0.13** | 5.17 ± 0.61** | 239.48 ± 53.92** |
| Blank | 100 | 19.90 ± 1.45 | 24.10 ± 2.13 | 0.25 ± 0.08 | 0.12 ± 0.04 | 1.63 ± 0.40 | 68.31 ± 16.68 |
| Emulsion + ⁶⁰Co | | | | | | | |
| ⁶⁰Co + Ganoderma | 1000 + 100 | 20.00 ± 1.63 | 24.60 ± 2.41 | 0.33 ± 0.08* | 0.14 ± 0.05 | 2.02 ± 0.41* | 85.60 ± 18.60* |
| lucidium | 500 + 100 | 19.70 ± 1.57 | 24.10 ± 2.13 | 0.29 ± 0.03 | 0.14 ± 0.03 | 2.03 ± 0.42* | 79.05 ± 16.51 |
| spore oil fat | 250 + 100 | 19.60 ± 1.07 | 24.30 ± 2.21 | 0.29 ± 0.05 | 0.12 ± 0.03 | 1.94 ± 0.54 | 79.93 ± 17.37 |
| emulsion for | | | | | | | |
| injection | | | | | | | |
| ⁶⁰Co + Thymosin | 0.42 + 100 | 19.60 ± 1.51 | 24.20 ± 2.25 | 0.33 ± 0.04* | 0.17 ± 0.07* | 1.98 ± 0.31* | 82.71 ± 13.58* |
| a1 | | | | | | | |

*P < 0.05,
**P < 0.01 Comparing with Blank Emulsion + ⁶⁰Co group.

Data from Table 9 demonstrate that Ganoderma lucidium spore oil fat emulsion i.v. dosage has toxicity reducing effects for chemotherapy with ⁶⁰Co in H₂₂ tumor bearing mice.

### Embodiment 21

Toxicity Reducing Effect of Ganoderma lucidium Spore Oil Fat Emulsion i.v . Dosage for Chemotherapy ⁶⁰Co in Normal Mice

Experiment Method: Take 60 ICR mice, apart from the 10 animals in blank control, the other 50 mice are radiated with ⁶⁰Co, with a dosage of 500 rad; after radiation with ⁶⁰Co, randomize the animals into 5 groups pursuant to body weight, with 10 animals in each group, half of which are females and half of which are males. In the experiment there are: Blank control group, Intravenous control group (20 ml/kg, Blank Emulsion), High dosage, Medium dosage and Low dosage group of Ganoderma lucidium spore oil fat emulsion for injection (1, 0.5, 0.25 g/kg) iv administered, Positive control drug Thymosin a1 injection group (0.42 mg/kg) Sc administered. All groups of animals are dosed 24 hours after innoculation, apart from that Thymosin a1 are dosed once every other day, the other groups are administered once daily, with a total dose of 7 times, and the mice are sacrificed 24 hours after termination of dosage, weigh and collect blood from the orbit veins before sacrificing, microscopically examine the total number of peripheral blood leukocytes; at the same time remove spleen, thymus gland and one complete femur, then determine the number of nucleated cells in bone marrow, and weigh the thymus gland and spleen, and calculate the coefficients of thymus glands and spleen organs, and carry out statistical treatment (t Test).

Experiment Results: Results demonstrate, compared with the blank control group, Peripheral blood leukocytes, and the number of nucleated cells in bone marrow as well as thymus gland and spleen coefficients in Blank Emulsion+⁶⁰Co model (500 rad) decline significantly (P<0.01). Compared with ⁶⁰Co model group, Thymosin a1 injection group could significantly withstand the decline in mice peripheral blood leukocytes, the number of nucleated cells in bone marrow, thymus gland coefficient and spleen coefficient (P<0.05) in normal mice caused by ⁶⁰Co, High dosage group of Ganoderma lucidium spore oil fat emulsion for injection could significantly withstand the decline of the number of peripheral blood leukocytes, the number of nucleated cells in bone marrow, and spleen coefficient (P<0.05) in normal mice caused by ⁶⁰Co, with the results demonstrated in Table 10.

TABLE 10
Toxicity reducing effect of Ganoderma lucidium spore oil fat
emulsion i.v. dosage for radiotherapy of ⁶⁰Co in normal mice ( X̅ ± SD, n = 10).

| | | | | | | | The number of |
|---|---|---|---|---|---|---|---|
| | | | | | Thymus | | nucleated cells |
| | | Body Weight (g) | | Spleen | Gland | | in bone |
| | Dosage | Before | After | Coefficient | Coefficient | WBC | marrow |
| Group | (mg/kg) | Dosage | Dosage | (g/100 g) | (g/100 g) | (10⁹/L) | (10⁵/piece) |
| Blank | | 19.60 ± 1.35 | 24.70 ± 2.11* | 0.50 ± 0.16** | 0.30 ± 0.13** | 4.27 ± 1.22** | 221.90 ± 63.09** |
| Control | | | | | | | |
| ⁶⁰Co + Blank | 100 | 19.40 ± 1.43 | 22.60 ± 2.12 | 0.18 ± 0.05 | 0.09 ± 0.04 | 0.87 ± 0.16 | 54.95 ± 14.22 |
| Emulsion | | | | | | | |
| 60Co + | 1000 + 100 | 19.70 ± 1.25 | 23.40 ± 2.12 | 0.26 ± 0.09* | 0.13 ± 0.06 | 1.09 ± 0.29* | 66.99 ± 11.21* |
| Ganoderma | 500 + 100 | 19.70 ± 1.42 | 22.60 ± 2.27 | 0.28 ± 0.12* | 0.09 ± 0.04 | 0.96 ± 0.35 | 63.04 ± 13.17 |
| lucidium | 250 + 100 | 19.50 ± 1.65 | 22.80 ± 2.25 | 0.28 ± 0.13* | 0.10 ± 0.04 | 0.98 ± 0.15 | 55.89 ± 13.43 |
| spore oil | | | | | | | |
| fat emulsion | | | | | | | |
| for injection | | | | | | | |
| ⁶⁰Co + | 0.42 + 100 | 19.90 ± 1.66 | 23.30 ± 2.11 | 0.31 ± 0.14* | 0.14 ± 0.05* | 1.21 ± 0.38* | 67.83 ± 13.09* |
| Thymosin | | | | | | | |
| a1 | | | | | | | |

*P < 0.05,
**P < 0.01 Compared with ⁶⁰Co + Blank Emulsion group.

Data from Table 10 demonstrate that Ganoderma lucidium spore oil fat emulsion i.v. dosage has toxicity reducing effects for radiotherapy with ⁶⁰Co in normal mice.

What is claimed is:

1. A kind of Fat emulsion of Ganoderma lucidium spore oil, characterized in that it is composed of the following ingredients and weight percentage:
refined Ganoderma lucidium spore oil: 2˜25%;
emulsifier: 0.5˜10%;
isosmotic agent: 0.2˜5%;
the remaining ingredient is water;
the final pH of Fat Emulsion mentioned above is between 6 and 9.

2. The Ganoderma lucidium spore oil fat emulsion as claimed in claim 1, is characterized in that the refined Ganoderma lucidium spore oil refers to the refining of Ganoderma lucidium spore oil after extraction, and the refining method is:
centrifuge the Ganoderma lucidium spore oil to eliminate the water content, add into adsorbent of 0.5%˜10% of Ganoderma lucidium spore oil weight, and then agitate till homogeneity, heated to 40˜70° C., keep the temperature constant for 20 to 40 minutes, then centrifuge, after film filtration, refined Ganoderma lucidium spore oil is obtained; the aforesaid adsorbents include one or several of the following: activated carbon, silica gel, Alumina N-neutral, diatomaceous earth, and carclazyte as the mixture.

3. Fat emulsion of Ganoderma lucidium spore oil as claimed in claim 1, is characterized in that the aforesaid emulsifiers are one or several of Soybean Lecithin, Yolk lecithin, pluronic, polyglycerol palmitic diol or alginates as the mixture.

4. Ganoderma lucidium spore oil fat emulsion as claimed in claim 1, is characterized in that the aforesaid isosmotic agents are one or several of the following: glycerin, glucose, mannitol, maltose or sorbitol as the mixture.

5. A quality control method for Ganoderma lucidium spore oil fat emulsion as claimed in claim 1, is characterized in that HPLC is adopted to determine the content of 1,2-oleic-3-palmitic triglyceride and/or Glycerol Trioleate, for every 1 g of Ganoderma lucidium spore oil fat emulsion, there contains 2 mg to 62.5 mg of 1,2-oleic-3-palmitic triglyceride and/or 1.6 mg to 50.0 mg Glycerol Trioleate;
HPLC mentioned above is carried out pursuant to the following
Chromatographic conditions: with octadecyl silane as the bulking agent, with the binary or ternary mixture of the three solvents acetonitrile, isopropanol, dichloromethane at any ratio as the mobile phase, the flow rate of mobile phase is 0.5˜2.0 ml/min; and then detected with evaporated light-scattering detector or differential refractive index detector; the number of theoretical plates is calculated pursuant to the peaks of 1,2-oleic3-palmitic triglyceride or Glycerol Trioleate, both of which shall be no lower than 2000;
with chromatographic column temperature between 10 and 50° C.

6. A quality control method for Ganoderma lucidium spore oil fat emulsion as claimed in claim 1, is characterized in that HPLC is adopted to determine the content of ergosterol, for every 1 g of Ganoderma lucidium spore oil fat emulsion, there contains 0.04 mg to 7.5 mg of ergosterol;
the chromatographic conditions are: With octadecyl silane as the bulking agent,
with methanol, ethanol, acetonitrile, methanol water solution, ethanol water solution or acetonitrile water solution as the mobile phase, or with ternary or quaternary mixture of methanol, ethanol, acetonitrile and water as the mobile phase, or with the mixture of tetrahydrofuran and water as the mobile phase, and the volume ratio between tetrahydrofuran and water is 75:25;
with the detecting wavelength of 280±2 nm; the number of theoretical plates is calculated pursuant to ergosterol peak, with the value no lower than 2000.

7. A quality control method for Ganoderma lucidium spore oil fat emulsion as claimed in claim 1, is characterized in that HPLC method is applied, through comparison between several batches of chromatogram for Ganoderma lucidium spore oil fat emulsion, and the standard fingerprints for Ganoderma lucidium spore oil fat emulsion, constituted by common characteristic peaks.

8. The standard fingerprints as claimed in claim 7, is characterized in that the HPLC conditions are: with octadecyl silane as the bulking agent for Chromatographic column; with acetonitrile-isopropanol as the mobile phase, and the volume ratio between them is 53:47; with the evaporative light-scattering detector; control: with Glycerol Trioleate as the control;
in the fingerprints of Ganoderma lucidium spore oil fat emulsion, there are 15
common peaks, among which there are 4 fingerprint peaks covering more than 5% of the total peak area respectively, with the relative retention time of Glycerol Trioleate chromatogram peak as 1, calculate the relative retention time of other chromatogrm peaks as well as the relative peak area, and the aforesaid 4 fingerprint peaks are respectively peak 9, with the average retention time RT as 0.778, and the relative peak area is between 9.54 and 15.36%; peak 10, with the average retention time RT as 0.832, and the relative peak area is between 5.76% and 9.43%; peak 11, with the average retention time RT for Glycerol Trioleate as 1.000, and the relative peak area between 22.29 and 27.80%; peak 12, with the average retention time RT as 1.075, and the relative peak area is between 26.82% and 37.76%.

9. A preparation of Ganoderma lucidium spore oil fat emulsion as claimed in claim 1, characterized in the application for the treatment of tumors while improving immunization, and reducing the toxicity of radiotherapy and chemotherapy drugs.

10. An application as claimed in claim 9, characterized in that the dosage form of the medicine mentioned above is fat emulsion for injection.
